(19) United States
(12) Patent Application Publication
(10) Pub. No.: US 2009/0264489 A1
(43) Pub. Date: Oct. 22, 2009

(54) Method for Treating Acute Pain with a Formulated Drug Depot in Combination with a Liquid Formulation

(75) Inventors: Keith R. HILDEBRAND, Houlton, WI (US); Christopher M. Hobot, Tonka Bay, MN (US); John Myers Zanella, Cordova, TN (US); Vanja Margareta King, Memphis, TN (US)

Correspondence Address: MEDTRONIC;Attn: Noreen Johnson - IP Legal Department, 2600 Sofamor Danek Drive, MEMPHIS, TN, 38132, US

(73) Assignees: Warsaw Orthopedic, Inc., Warsaw, IN (US); Medtronic, Inc., A Minnesota Corporation, Minneapolis, MN (US)

(21) Appl. No.: 12/397,869

(22) Filed: Mar. 4, 2009

Related U.S. Application Data
(60) Provisional application No. 61/153,817, filed on Feb. 19, 2009.
(60) Provisional application No. 61/046,201, filed on Apr. 18, 2008.

(51) Int. Cl. A61K 31/4168; A61P 25/00

(52) U.S. Cl. 514/401

(57) ABSTRACT

Effective methods for treating pain are provided. Through the injection of liquid formulation comprising an active ingredient followed by the administration of drug depot comprising an active ingredient at or near a target site, one can effectively treat pain. This methodology is particularly effective to treat acute radicular pain.

This application claims the benefit of the filing date of U.S. Provisional Patent Application No. 61/046,201 filed Apr. 18, 2008, entitled “Clonidine Formulations In A Biodegradable Polymer Carrier” and U.S. Provisional Patent Application No. 61/153,817 filed Feb. 19, 2009, entitled “Method for Treating Acute Pain with a Formulated Drug Depot in Combination with a Liquid Formulation.” These entire disclosures are hereby incorporated by reference into the present disclosure.

### BACKGROUND

Pain is typically experienced when the free nerve endings of pain receptors are subject to mechanical, thermal, chemical or other noxious stimuli. These pain receptors can transmit signals along afferent neurons to the central nervous system and then to the brain. When a person feels pain, any one or more of a number of problems can be associated with this sensation, including but not limited to reduced function, reduced mobility, complication of sleep patterns, and decreased quality of life.

One known type of pain is acute radicular pain, which refers to pain that radiates along the dermatome of a nerve. This pain may be due to inflammation or other irritation of the nerve root at its connection to the spinal column. A common form of radiculitis is sciatica, or radicular pain that radiates along the sciatic nerve from the lower spine to the lower back, gluteal muscles, back of the upper thigh, calf, and foot as often secondary to nerve root irritation from a spinal disc herniation or from bone spurs or ostophytes in the lumbar region of the spine.

The causes of pain include but are not limited to inflammation, injury, disease, muscle stress, the onset of a neuropathic event or syndrome, and damage that can result from surgery or an adverse physical, chemical or thermal event or from infection by a biologic agent. When a tissue is damaged, a host of endogenous pain inducing substances, for example, bradykinin and histamine can be released from the injured tissue. The pain inducing substances can bind to receptors on the sensory nerve terminals and thereby initiate afferent pain signals. After activation of the primary sensory afferent neurons, the projection neurons may be activated. These neurons carry the signal via the spinothalamic tract to higher parts of the central nervous system.

There have been numerous proposals for which active ingredients are most effective for treating pain, including acute radicular pain. However, generally these treatments focus on individual active-ingredients that are administered in only one type of formulation. Because different active ingredients, and different vehicles for administering them have different benefits and risks, there is a need to develop better methods for administering different active ingredients in different vehicles to treat pain at the same target site.

### SUMMARY

Methods are provided comprising administering a liquid formulation and a drug depot to treat pain at a target site. According to one embodiment, there is a method for treating a mammal suffering from pain, said method comprising injecting a therapeutically effective amount of a liquid formulation at or near a target site, wherein said liquid formulation comprises a first active ingredient that is effective at treating pain, and after injecting said liquid formulation, injecting a drug depot locally at or near the target site, wherein the drug depot comprises a therapeutically effective amount of a second active ingredient. The first and second active ingredients may be the same or different from each other. For example, the first active ingredient may be a steroid and the second active ingredient may be clonidine or both active ingredients may be clonidine. In some embodiments, one or both of the active ingredients is a analgesic.

According to another embodiment, there is a method for treating a mammal suffering from pain, the method comprising injecting a therapeutically effective amount of a liquid formulation at or near a target site, wherein said liquid formulation comprises clonidine and/or a steroid that is effective at treating pain, and after injecting said liquid formulation, administering a drug depot at or near the target site, wherein the drug depot comprises a therapeutically effective amount of clonidine in a formulation that provides for relief from pain for a period of at least three days.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

In part, other aspects, features, benefits and advantages of the embodiments will be apparent with regard to the following description, appended claims and accompanying drawings where:

FIG. 1 illustrates a number of common locations within a patient that may be sites at which pain may occur and locations at which both the liquid formulation and the drug depot can locally be administered thereto and used to treat pain.

FIG. 2 illustrates a schematic dorsal view of the spine and sites where the injectable formulation and the drug depot can locally be administered thereto.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth are reported as precisely as possible. Any numerical value, however, inherently contains certain errors necessarily resulting from the standard deviation found in its respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

Definitions

Unless otherwise specified or apparent from context, the following terms and phrases have the meanings specified below.

It is noted that, as used in this specification and the appended claims, the singular forms “a,” “an,” and “the,” include plural referents unless expressly and unequivocally limited to one referent. Thus, for example, reference to “a drug depot” includes one, two, three or more drug depots.

A “depot” includes but is not limited to capsules, (micro)spheres, (micro)particles, (micro)capsules, (micro)fibers particles, nanospheres, nanoparticles, coating, matrices, wafers, sheets, strips, ribbon-like fibers, ribbons, pills, pellets, emulsions, liposomes, micelles, gels, or other pharmaceutical delivery compositions or a combination thereof. Suitable materials for the depot are ideally pharmaceutically acceptable biodegradable and/or any bioabsorbable materials that are preferably FDA approved or GRAS materials. These materials can be polymeric or non-polymeric, as well as synthetic or naturally occurring, or a combination thereof. In some embodiments, a depot is a solid composition.

The term “biodegradable” includes that all or parts of the drug depot will degrade over time by the action of enzymes, by hydrolytic action and/or by other similar mechanisms in the human body. In various embodiments, “biodegradable” includes that the depot (e.g., microparticle, microsphere, etc.) can break down or degrade within the body to non-toxic components after or while a therapeutic agent has been or is being released. By “bioerodible” it is meant that the depot will erode or degrade over time due, at least in part, to contact with substances found in the surrounding tissue, fluids or by cellular action. By “bioabsorbable” it is meant that the depot will be broken down and absorbed within the human body, for example, by a cell or tissue. “Biocompatible” means that the depot will not cause substantial tissue irritation or necrosis at the target tissue site.

The phase “drug depot” as used herein refers to a composition in which a therapeutic agent such as clonidine may be administered to the body and implanted in the body. Thus, a drug depot may comprise a physical structure to facilitate implantation and retention in a desired site (e.g., a disc space, a spinal canal, a tissue of the patient, particularly at or near a site of surgery, etc.). The drug depot also comprises the drug itself. The term “drug” as used herein is generally meant to refer to any substance that alters the physiology of a patient. The term “drug” may be used interchangeably herein with the terms “therapeutic agent,” “therapeutically effective amount,” and “active pharmaceutical ingredient” or “API.” It will be understood that unless otherwise specified a “drug” formulation may include more than one therapeutic agent, wherein exemplary combinations of therapeutic agents include a combination of two or more drugs. The drug provides a concentration gradient of the therapeutic agent for delivery to the site. In various embodiments, the drug depot provides an optimal drug concentration gradient of the therapeutic agent at a distance of up to about 0.1 cm, up to about 1 cm, up to about 2 cm, up to about 3 cm, up to about 4 cm or up to about 5 cm from the implant site, and comprises clonidine.

The phrase “immediate release” is used herein to refer to one or more therapeutic agent(s) that is introduced into the body and that is allowed to dissolve in or become absorbed at the location to which it is administered, with no intention of delaying or prolonging the dissolution or absorption of the drug. For example, in some embodiments, a liquid formulation comprising an immediate release active ingredient to treat pain can be administered at or near a target tissue site. The immediate release liquid formulation can release all or substantially all of the active ingredient(s) within a 24 hours period. For example, the immediate release formulation can be active for 1-12 hours, 4-6 hours 2-3 hours or until the drug depot begins to release its active ingredient(s).

In various embodiments, when the depot is a gel, the drug depot can be designed to cause an initial burst dose of therapeutic agent within the first 24 hours after implantation. “Initial burst” or “burst effect” or “bolus dose” refers to the release of therapeutic agent from the depot during the first 24 hours after the depot comes in contact with an aqueous fluid (e.g., synovial fluid, cerebral spinal fluid, etc.). The “burst effect” is believed to be due to the increased release of therapeutic agent from the depot (e.g., gel) while it is coagulating or hardening to form a solid or semi solid (rubbery) implant, while the gel is still in a flowable state. In alternative embodiments, the depot (e.g., gel) is designed to avoid this initial burst effect. The fluid may be used to induce a limited burst from depot in order to achieve better initial pain relief in the case of a double API system. This could be accomplished by adding an agent to the fluid phase that increases the solubility of the drug contained in the polymer, such as ethanol or a surfactant.

The phrase “localized delivery” includes delivery where one or more drugs are deposited within a tissue, or near a nerve root of the nervous system or a region of the brain, or in close proximity (within about 10 cm, or preferably within about 5 cm, for example) thereto. The phrase “targeted delivery system” refers to providing delivery of one or more drugs depots, gels or depot dispersed in the gel having a quantity of therapeutic agent that can be deposited at or near the target site as needed for treatment of pain, inflammation or other disease or condition. The term “locally” refers to a proximity to the site of interest such that when the drug is released, an effective amount of the clonidine will reach the site.

The term “mammal” refers to organisms from the taxonomy class “mammalian,” including but not limited to humans, other primates such as chimpanzees, apes orangutans and monkeys, rats, mice, cats, dogs, cows, horses, etc.

The term “pain management medication” includes one or more therapeutic agents that are administered to prevent, alleviate or remove pain entirely. These include anti-inflammatory agents, muscle relaxants, analgesics, anesthetics, narcotics, and so forth, and combinations thereof.

The phrase “release rate profile” refers to the percentage of active ingredient that is released over fixed units of time, e.g., mcg/hr, mcg/day, 10% per day for ten days, etc. As persons of ordinary skill know a release rate profile may be but need not be linear. By way of a non-limiting example, the drug depot may be a ribbon-like fiber that releases the clonidine over a period of time.

The term “solid” is intended to mean a rigid material, while, “semi-solid” is intended to mean a material that has some degree of flexibility, thereby allowing the depot to bend and conform to the surrounding tissue requirements.

The phrases “sustained release” and “sustain release” (also referred to as extended release or controlled release) are used herein to refer to one or more therapeutic agent(s) that are introduced into the body of a human or other mammal and continuously or intermittently releases an amount of one or more therapeutic agents over a predetermined time period and at a therapeutic level sufficient to achieve a desired therapeutic effect throughout the predetermined time period. Reference to a continuous release stream is intended to encompass release that occurs as the result of biodegradation in vivo of the drug depot, or a matrix or component thereof, or as the result of metabolic transformation or dissolution of the therapeutic agent(s) or conjugates of therapeutic agent(s).

The phrase “target site” refers to the site within an organism at which pain is either caused or felt. A treatment that is administered locally at or near a target site, is administered within a distance that permits the active ingredient to cause the desired result at the target site.

The terms “treating” or “treatment” with respect to a disease or condition refers to executing a protocol, which may include administering one or more drugs to a patient (human, either normal or otherwise), in an effort to alleviate signs or symptoms of the disease. Alleviation can occur prior to signs or symptoms of the disease or condition appearing, as well as after their appearance. Thus, “treating” or “treatment” includes “preventing” or “prevention” of disease or undesirable condition. In addition, “treating” or “treatment” does not require complete alleviation of signs or symptoms, does not require a cure, and specifically includes protocols that have only a marginal effect on the patient. The compositions and methods provided may be used to reduce, prevent, or treat inflammation and/or pain, including but not limited to inflammation and/or pain that follows surgery, chronic inflammatory diseases, chronic inflammatory bowel disease, chronic pelvic pain conditions such as painful bladder syndrome and chronic prostatits, osteoarthritis, osteolysis, tendonitis, sciatica, herniated discs, stenosis, myopathy, spondilothesis, lower back pain, facet pain, carpal tunnel syndrome, tarsal tunnel syndrome, failed back pain or the like.

A “therapeutically effective amount” or “effective amount” is such that when administered, the drug results in alteration of the biological activity, such as, for example, inhibition of inflammation, reduction or alleviation of pain or spasticity, improvement in the condition through muscle relaxation, etc. The dosage administered to a patient can be as single or multiple doses depending upon a variety of factors, including the drug's administered pharmacokinetic and pharmacodynamic properties, the route of administration, patient conditions and characteristics (sex, age, body weight, health, size, etc.), extent of symptoms, concurrent treatments, frequency of treatment and the effect desired.

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents that may be included within the invention as defined by the appended claims.

The headings below are not meant to limit the disclosure in any way; embodiments under any one heading may be used in conjunction with embodiments under any other heading.

The present invention provides methods for treating pain such as acute radicular or pain associated with stenosis, and tendonitis through the two steps process of administering a liquid formulation containing a first active ingredient, and administering a drug depot containing a second active ingredient. Both the liquid formulation and the drug depot may be administered locally at a pain site. In some embodiments, the liquid formulation may provide immediate relief from pain, whereas the drug depot may provide a sustained release that begins immediately upon implantation or after a certain amount of time that is dependent upon the formulation of the drug depot. An advantage of the two part system is that the fluid phase treats the acute pain and avoids the need for a burst from the depot. This allows the drug stored in the depot to be reserved for providing sustained efficacy thereby increasing the total amount of time the drug is released and pain is treated.

Further and as described more fully below in some embodiments, the liquid formulation may physically be introduced into the patient at a first time, and the drug depot may be introduced into the patient at a second later time, that is immediately after the introduction of the liquid formulation or after a specific time interval. In other embodiments, the liquid formulation is introduced into the patient either at the same time as the drug depot or after the drug depot, but because of the formulation of drug depot, the active ingredient is released over and extended time, whereas the liquid formulation provides for immediate release. The methods of the present invention may also be used to treat neuropathic pain and inflammatory pain.

The Liquid Formulation

The liquid formulation may be designed to provide immediate relief from pain and in some embodiments may be injected. For example, the liquid formulation may be an epidural steroid injection, an epidural clonidine injection or an epidural injection of both clonidine and a steroid. Other exemplary liquid formulations may include one or more of lidocaine, bupivacaine, and NSAIDs such ketorolac tromethamine.

Exemplary steroids that may be of use in connection with the present invention include but are not limited to glucocorticoids, angiostatic, and corticosteroids. Suitable glucocorticoids that can be employed include, but are not limited to, dexamethasone, fluoromethalone, medrysone, betamethasone, triamcinolone, triamcinolone acetonide, prednisone, prednisolone, hydrocortisone, rimexolone, prednicarbate, deflazacort, halomethasone, tixocortol, prednylidene, prednival, paramethasone, methylprednisone, meprednisone, mazipredone, isoflupredone, halopredone acetate, halcinonide, formocortal, flurandrenolide, fluprednisone, fluprednidine acetate, fluperolone acetate, fluocortolone, fluocortin butyl, fluocinonide, fluocinolone acetonide, flunisolide, flumethasone, fludrocortisone, fluclorinide, enoxolone, difluprednate, diflucortolone, diflorasone diacetate, desoximetasone, desonide, descinolone, cortivazol, corticosterone, cortisone, cloprednol, clocortolone, clobetasone, clobetasol, chloroprednisone, cafestol, budesonide, beclomethasone, amcinonide, allopregnane acetonide, aldlometasone, 21-acetoxypregnenolone, tralonide, diflorasone acetate, deacylcortivazol, RU-26988, and deacyulcortivazol oxetanone. The steroid can also be a pharmaceutically acceptable salt of any of the foregoing, in which case the steroid salt is preferably insoluble, or more preferably sparingly soluble, in water.

Exemplary angiostatic steroids include but are not limited to hydrocortisone, tetrahydrocortisol-S,11α-epihydrocotisol, cortexolone, 17α-hydroxy-progesterone, corticosterone, deoxycorticosterone, testosterone, estrone, dexamethasone, 6α-fluoro-17,21-dihydroxy-16β-methyl-pregna-4,9,(11)-diene-3,20-dione and anecortave acetate.

As used herein, a “steroid” includes a steroid or its pharmaceutically acceptable salts; pharmacologically-active derivatives of the steroid or an active metabolite of the steroid. As used herein, “pharmaceutically acceptable salts” refer to derivatives of the disclosed compounds (e.g., esters or amines) wherein the parent compound may be modified by making acidic or basic salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, or nitric acids; or the salts prepared from organic acids such as acetic, fuoric, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic, isethionic acid. Pharmaceutically acceptable also includes the racemic mixtures ((+)-R and (−)-S enantiomers) or each of the dextro and levo isomers of the steroid individually. The steroid may be in the free acid or base form or be pegylated for long acting activity.

The one or more steroids may for example have an average particle size of from about 2.2 to about 10 microns. In some embodiments the steroid particles have a minimum average particle size of about 2.2 microns, or about 2.5 microns, or about 3 microns, or about 4 microns. The particles also may have a maximum average particle size of about 10 microns, or about 8 microns, or about 7 microns, or about 5 microns. Moreover, the steroid particles may have a monophasic distribution. Additionally, in some embodiments, it may be preferable to have a water-soluble steroid in order to produce an acute anti-inflammatory/analgesic effect that the depot is not providing.

The unit dose vial for the application of a liquid formulation containing a steroid preferably contains enough steroid to be therapeutically effective for a human, and the indication to be treated can be any suitable condition. In certain preferred applications, the single unit dose vial or preloaded syringe of the pharmaceutical composition of the invention is suitable for use in administering the composition to either the cerebrospinal system, or to the musculoskeletal system. The pharmaceutical composition may be administered in a total volume of about 10 microliters to about 2 ml, preferably about 100 microliters to about 1 ml. The dose may also have a total volume of about 50 microliters or less. The dose may preferably have a total volume of or up to about 10 microliters, 15 microliters, 20 microliters, 25 microliters, 30 microliters, 35 microliters, 40 microliters, 45 microliters, 50 microliters, 55 microliters, 60 microliters, 65 microliters, 70 microliters, 75 microliters, 80 microliters, 85 microliters, 90 microliters, 95 microliters, 100 microliters, 200 microliters, 300 microliters, 400 microliters, 500 microliters, 600 microliters, 700 microliters, 800 microliters, 900 microliters, or 1 ml or intermediate dosages. The dose may have a total volume greater than 1 ml, such as 1.1 ml, 1.2 ml, 1.3 ml, 1.4 ml, 1.5 ml, 1.6 ml, 1.7 ml, 1.8 ml, 1.9 ml, 2 ml, or more than about 2 ml, as well as intermediate dosages. The pharmaceutical composition is preferably administered in a single injection or, alternatively, in multiple injections, wherein multiple unit doses may be administered to the patient at the discretion of the treating physician based on the patient's size, medical condition, or other relevant criteria in determining the appropriate dosage. Preferably, a patient will receive a single dose. In some cases, a patient may receive multiple doses in a single treatment. In some embodiments, the liquid may be administered within four hours before implanting the drug depot or within 5 minutes, within 10 minutes, within 15 minutes, within 20 minutes, within 25 minutes, within 30 minutes, within 35 minutes, within 40 minutes, within 45 minutes, within 50 minutes, within 55 minutes, or within 60 minutes of implanting the drug depot.

In some embodiments from about 10 micrograms to about 160 micrograms of steroid are administered through one or more injections in a solution, for example an aqueous form. In some embodiments from about 10 micrograms to about 30 micrograms of steroid are administered through one or more injections. In some embodiments from about 30 micrograms to about 50 micrograms of steroid are administered through one or more injections. In some embodiments from about 50 micrograms to about 70 micrograms of steroid are administered through one or more injections. In some embodiments from about 70 micrograms to about 90 micrograms of steroid are administered through one or more injections. In some embodiments from about 90 micrograms to about 110 micrograms of steroid are administered through one or more injections. In some embodiments from about 110 micrograms to about 130 micrograms of steroid are administered through one or more injections. In some embodiments from about 130 micrograms to about 160 micrograms of steroid are administered through one or more injections.

When the liquid formulation comprises clonidine, the clonidine may for example be in a formulation that is approximately 0.1 to 0.5 mg/mL or 0.5 to 1.0 mg/mL or 1.0 to 2.0 mg/mL of clonidine hydrochloride in water, e.g., 1.5 mg/mL of clonidine hydrochloride. Exemplary dosages of injectable clonidine may for example contain the following amounts of clonidine: from about 10 micrograms to about 20 micrograms, from about 20 micrograms to about 30 micrograms and from about 30 micrograms to about 40 micrograms.

As persons of ordinary skill in the art are aware, epidural clonidine may produce an analgesic effect by preventing the pain-signal transmission to the brain at presynaptic and postjunctional alpha-2 adrenoceptors in the spinal cord.

Further, other alpha 2-adrenergic agonists may be used as an active ingredient instead of clonidine or in combination with it. Examples of alpha-2 adrenergic receptor agonists useful in the present application include, but are not limited to L-norepinephrine, dexmetdetomidine, apraclonidine, methyldopa, tizanidine, brimonidine, xylometazoline, tetrahydrozoline, oxymetazoline, guanfacine, guanabenz, guanoxabenz, guanethidine, xylazine, moxonidine, mivazerol, rilmenidine, UK 14,304, B-HT 933, B-HT 920, octopamine or a combination thereof.

Unless otherwise specified or apparent from context, where this specification and the set of claims that follows refer to clonidine, the inventors are also referring to a pharmaceutically acceptable salts of clonidine. Some examples of pharmaceutically acceptable salts include those salt-forming acids and bases that do not substantially increase the toxicity of the compound. Some examples of suitable salts include salts of alkali metals such as magnesium, potassium and ammonium. Salts of mineral acids such as hydrochloric, hydriodic, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g., p-toluenesulfonic acids, and the like. Exemplary formulations may use either the base or the hydrochloric salt of clonidine.

According to some embodiments, only a single dose of the liquid formulation is given. According to other embodiments, multiple doses of the liquid formulation may be given. When multiple doses are give, they may for example, be given over about 1 to about 24 hours or about 1 to about 2 hours, or about 2 hours to about 3 hours, or about 3 hours to about 4 hours, or about 4 hours to about 5 hours or about 5 hours to about 6 hours or about 6 hours to about 7 hours or about 7 hours to about 8 hours or about 8 hours to about 9 hours or about 9 hours to about 10 hours or about 10 hours to about 11 hours or about 11 hours to about 12 hours or about 12 hours to about 13 hours or about 13 hours to about 14 hours or about 14 hours to about 15 hours or about 15 hours to about 16 hours or about 16 hours to about 17 hours or about 17 hours to about 18 hours or about 18 hours to about 19 hours or about 19 hours to about 20 hours or about 20 hours to about 21 hours or about 21 hours to about 22 hours or about 22 hours to about 23 hours or about 23 hours to about 24 hours.

In some embodiments, the liquid formulation is free from classical preservatives and/or free of dispersion agents. In some embodiments, the liquid formulation comprises, consists of or consists essentially of the active ingredient (or its pharmaceutically acceptable salt), water and optionally a suitable excipient.

Exemplary excipients for the liquid formulation include but are not limited to methylcellulose, hydroxypropylmethylcellulose, hydroxyethylcellulose, polyvinyl alcohol, and polyethylene glycol. In some embodiments, the liquid formulation may for example contain a minimum excipient concentration of at least about 0.2%, or at least about 0.35%, or at least about 0.5%, wherein the percentages are measured in weight per volume. Additionally, in some embodiments, the liquid formulation may contains a maximum excipient concentration of about 5%, or about 2%, or about 1% excipient, wherein these percentages are measured in weight per volume.

In some embodiments, when the active ingredient in the liquid formulation is a steroid, the steroid and excipient may be carried by an aqueous carrier, which may be a combination of a salt and water. Any suitable salt can be employed; however, the salt should be acceptable for pharmaceutical use in the concentration employed and is more preferably suitable for cerebral spinal systems and/or musculoskeletal use in the concentration employed. The salt may for example be sodium chloride. The pharmaceutical composition preferably contains at least about 0.7% (w/v) sodium chloride and no more than about 1.1% (w/v) sodium chloride (e.g., about 0.8-1% (w/v)). More preferably, the pharmaceutical composition contains about 0.9% sodium chloride. Additionally, the salt concentration or excipient concentration or both are preferably adjusted, if necessary, to provide an osmolarity of from about 200 mOsm to about 400 mOsm.

In some embodiments, the active ingredient (e.g., steroid, clonidine, etc.) may be in powder form and can be reconstituted with one or more liquid diluents that may be aqueous. For example, an aqueous liquid diluent may be water, pharmaceutically acceptable aqueous solutions, aqueous saline solutions (NS, ½ NS, etc.), Ringer's solutions, lactated Ringer's solutions, bicarbonate solutions, or aqueous dextrose solutions, or combinations thereof. The liquid diluent may contain one or more excipients such as the antioxidant BHT (butylated hydroxytoluene).

In another embodiment, the liquid diluent is non-aqueous and comprises one or more surfactants, e.g., non-ionic surfactants. In general, the weight to weight ratio (w/w) between the active ingredient or a salt thereof and the non-ionic surfactant(s) may be from about 1:10,000 to about 1:1. Useful non-ionic surfactants can include a polyethoxylated castor oil, a polysorbate, a sorbitan ester, a polyoxyethylene fatty acid ester, a polyoxyethylene fatty acid ether, a polyoxyethylene alkyl ether, and an ethoxylated fatty acid.

In other embodiments, a liquid diluent may be a combination of aqueous diluents and non-aqueous diluents. For example, a non-aqueous liquid diluent comprising one or more non-ionic surfactants may further include an aqueous diluent, such as water, pharmaceutically acceptable aqueous solutions, aqueous saline solutions, Ringer's solutions, lactated Ringer's solutions, bicarbonate solutions, aqueous dextrose solutions, or combinations thereof. In a specific embodiment, the volume to volume ratio (v/v) of non-ionic surfactant to aqueous diluent may be from about 100:1 to about 1:20,000.

When the liquid formulation comprise both the steroid and the clonidine, a fraction of each of the above-described formulation may be combined in the same syringe or co-administered through different syringes.

The Drug Depot

In some embodiments, the drug depot comprises one or more of clonidine, decadron, bupivacaine or lidocaine or other agent for relieving pain such as analgesics that include but are not limited to, acetaminophen, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, mepiridine, methadone, morphine, nalbuphine, opium, oxycodone, papaveretum, pentazocine, pethidine, phenoperidine, piritramide, dextropropoxyphene, remifentanil, tilidine, tramadol, codeine, dihydrocodeine, meptazinol, dezocine, eptazocine, flupirtine or a combination thereof. Analgesics also include agents with analgesic properties, such as for example, amitriptyline, carbamazepine, gabapentin, pregabalin, or a combination thereof.

The drug depot may be co-administered with a muscle relaxant. Exemplary muscle relaxants include by way of example and not limitation, alcuronium chloride, atracurium bescylate, baclofen, carbolonium, carisoprodol, chlorphenesin carbamate, chlorzoxazone, cyclobenzaprine, dantrolene, decamethonium bromide, fazadinium, gallamine triethiodide, hexafluorenium, meladrazine, mephensin, metaxalone, methocarbamol, metocurine iodide, pancuronium, pridinol mesylate, styramate, suxamethonium, suxethonium, thiocolchicoside, tizanidine, tolperisone, tubocuarine, vecuronium, or combinations thereof.

The drug depot may further comprise other therapeutic agents. Therapeutic agents, in various embodiments, block the transcription or translation of TNF-α or other proteins in the inflammation cascade. Suitable therapeutic agents include, but are not limited to, integrin antagonists, alpha-4 beta-7 integrin antagonists, cell adhesion inhibitors, interferon gamma antagonists, CTLA4-Ig agonists/antagonists (BMS-188667), CD40 ligand antagonists, Humanized anti-IL-6 mAb (MRA, Tocilizumab, Chugai), HMGB-1 mAb (Critical Therapeutics Inc.), anti-IL2R antibodies (daclizumab, basilicimab), ABX (anti IL-8 antibodies), recombinant human IL-10, or HuMax IL-15 (anti-IL 15 antibodies), NF kappa B inhibitors such as glucocorticoids, antioxidants, such as dilhiocarbamate, and other compounds, such as, for example, sulfasalazine, IL-1 inhibitors, such Kineret® (anakinra) which is a recombinant, non-glycosylated form of the human inerleukin-1 receptor antagonist (IL-1Ra), or AMG 108, which is a monoclonal antibody that blocks the action of IL-1, excitatory amino acids such as glutamate and aspartate, antagonists or inhibitors of glutamate binding to NMDA receptors, AMPA receptors, and/or kainate receptors, interleukin-1 receptor antagonists, thalidomide (a TNF-α release inhibitor), thalidomide analogues (which reduce TNF-α production by macrophages), bone morphogenetic protein (BMP) type 2 and BMP-4 (inhibitors of caspase 8, a TNF-α activator), quinapril (an inhibitor of angiotensin II, which upregulates TNF-α), interferons such as IL-11 (which modulate TNF-α receptor expression), pentoxifylline (a small molecule TNF inhibitor) and aurin-tricarboxylic acid (which inhibits TNF-α). It is contemplated that where desirable a pegylated form of the above may be used.

Classes of therapeutic agents that may be used in conjunction with the aforementioned active ingredients include, but are not limited to an anti-inflammatory agents, or osteoinductive growth factors or a combination thereof. Anti-inflammatory agents include, but are not limited to, salicylates, diflunisal, sulfasalazine, indomethacin, ibuprofen, naproxen, tolmetin, ketorolac, diclofenac, ketoprofen, fenamates (mefenamic acid, meclofenamic acid), enolic acids (piroxicam, meloxicam), nabumetone, celecoxib, etodolac, nimesulide, apazone, gold, sulindac or tepoxalin; antioxidants, such as dithiocarbamate, and other compounds such as sulfasalazine [2-hydroxy-5-[-4-[C2-pyridinylamino)sulfonyl]azo]benzoic acid], steroids, such as fluocinolone, cortisol, cortisone, hydrocortisone, fludrocortisone, prednisone, prednisolone, methylprednisolone, triamcinolone, betamethasone, dexamethasone, beclomethasone, fluticasone or a combination thereof.

Suitable anabolic growth or anti-catabolic growth factors include, but are not limited to, a bone morphogenetic protein, a growth differentiation factor, a LIM mineralization protein, CDMP or progenitor cells or a combination thereof.

The active ingredient of the drug depot may also be administered with non-active ingredients. These non-active ingredients may have multi-functional purposes including the carrying, stabilizing and controlling the release of the therapeutic agent(s), i.e., giving them sustained release properties. The sustained release process, for example, may be by a solution-diffusion mechanism or it may be governed by an erosion-sustained process. Typically, the depot will be a solid or semi-solid formulation comprised of a biocompatible material, which can be biodegradable.

In various embodiments, the non-active ingredients will be durable within the tissue site for a period of time equal to (for biodegradable components) or greater than (for non-biodegradable components) the planned period of drug delivery. For example, the depot material may have a melting point or glass transition temperature close to or higher than body temperature, but lower than the decomposition or degradation temperature of the therapeutic agent. However, the pre-determined erosion of the depot material can also be used to provide for slow release of the loaded therapeutic agent(s).

Suitable drug depots for use in the present application are described in U.S. Provisional Application No. 61/046,246 filed Apr. 18, 2008, U.S. Provisional Application No. 61/046,218 filed Apr. 18, 2008, U.S. Provisional Application No. 61/046,218 filed Apr. 18, 2008, U.S. Provisional Application No. 61/046,201 filed Apr. 18, 2008, U.S. Ser. No. 12/105,864 filed Apr. 18, 2008 and U.S. Ser. No. 12/105,375 filed Apr. 18, 2008. The entire disclosure of these applications is herein incorporated by reference into the present application.

### Exemplary Pharmaceutical Active Ingredients Fluocinolone

In one embodiment, the anti-inflammatory agent is in liquid form and/or in the drug depot and comprises fluocinolone or a pharmaceutically acceptable salt thereof such as the acetonide salt. Fluocinolone is available from various pharmaceutical manufacturers. The dosage of fluocinolone may be from approximately 0.0005 to approximately 100 μg/day. Additional dosages of fluocinolone include from approximately 0.0005 to approximately 50 μg/day; approximately 0.0005 to approximately 25 μg/day; approximately 0.0005 to approximately 10 μg/day; approximately 0.0005 to approximately 5 μg/day; approximately 0.0005 to approximately 1 μg/day; approximately 0.0005 to approximately 0.75 μg/day; approximately 0.0005 to approximately 0.5 μg/day; approximately 0.0005 to approximately 0.25 μg/day; approximately 0.0005 to approximately 0.1 μg/day; approximately 0.0005 to approximately 0.075 μg/day; approximately 0.0005 to approximately 0.05 μg/day; approximately 0.001 to approximately 0.025 μg/day; approximately 0.001 to approximately 0.01 μg/day; approximately 0.001 to approximately 0.0075 μg/day; approximately 0.001 to approximately 0.005 μg/day; approximately 0.001 to approximately 0.025 μg/day; and approximately 0.002 μg/day. In another embodiment, the dosage of fluocinolone is from approximately 0.001 to approximately 15 μg/day. In another embodiment, the dosage of fluocinolone is from approximately 0.001 to approximately 10 μg/day. In another embodiment, the dosage of fluocinolone is from approximately 0.001 to approximately 5 μg/day. In another embodiment, the dosage of fluocinolone is from approximately 0.001 to 2.5 μg/day. In some embodiments, the amount of fluocinolone is between 40 and 600 μg/day. In some embodiments, the amount of fluocinolone is between 200 and 400 μg/day.

### Dexamethasone

In one embodiment the anti-inflammatory agent is in liquid form and/or in the drug depot and comprises dexamethasone free base or dexamethasone acetate, also referred to as 8S,9R,10S,11S,13S,14S,16R,17R)-9-Fluoro-11,17-dihydroxy-17-(2-hydroxyacetyl)-10,13,16-trimethyl-6,7,8,11,12,14,15,16octahydrocyclopenta[a]-phenanthren-3-one), or a pharmaceutically acceptable salt thereof, which is available from various manufacturers.

In various embodiments, dexamethasone may be released from the depot at a dose of about 10 μg to about 80 mg/day, about 2.4 ng/day to about 50 mg/day, about 50 ng/day to about 2.5 mg/day, about 250 ng/day to about 250 ug/day, about 250 ng/day to about 50 ug/day, about 250 ng/day to about 25 ug/day, about 250 ng/day to about 1 ug/day, about 300 ng/day to about 750 ng/day or about 0.50 ug/day. In various embodiments, the dose may be about 0.01 to about 10 mg/day or about 1 ng to about 120 mg/day. In one exemplary embodiment, the dexamethasone is dexamethasone sodium phosphate.

### GED

In one embodiment the therapeutic agent is in liquid form and/or in the drug depot and comprises GED (guanidinoethyldisulfide), which is an inducible nitric oxide synthase inhibitor having anti-inflammatory properties. GED may be in its hydrogen carbonate salt form.

The dosage of GED may be from approximately 0.0005 μg/day to approximately 100 mg/day. Additional dosages of GED include from approximately 0.0005 μg/day to approximately 50 mg/day; approximately 0.0005 μg/day to approximately 10 mg/day; approximately 0.0005 μg/day to approximately 1 mg/day; approximately 0.0005 to approximately 800 μg/day; approximately 0.0005 to approximately 50 μg/day; approximately 0.001 to approximately 45 μg/day; approximately 0.001 to approximately 40 μg/day; approximately 0.001 to approximately 35 μg/day; approximately 0.0025 to approximately 30 μg/day; approximately 0.0025 to approximately 25 μg/day; approximately 0.0025 to approximately 20 μg/day; and approximately 0.0025 to approximately 15 μg/day. In another embodiment, the dosage of GED is from approximately 0.005 to approximately 15 μg/day. In another embodiment, the dosage of GED is from approximately 0.005 to approximately 10 μg/day. In another embodiment, the dosage of GED is from approximately 0.005 to approximately 5 μg/day. In another embodiment, the dosage of GED is from approximately 0.005 to 2.5 μg/day. In some embodiments, the amount of GED is between 40 and 600 μg/day. In some embodiments, the amount of GED is between 200 and 400 μg/day.

In one exemplary embodiment the dosage of GED is between 0.5 and 4 mg/day. In another exemplary embodiment the dosage of GED is between 0.75 and 3.5 mg/day.

### Lovastatin

In one embodiment the anti-inflammatory agent is in liquid form and/or in the depot and comprises lovastatin. Lovastatin is a statin that may be obtained from various manufacturers in various forms (e.g., injection, powder, etc.). For example, lovastatin may be obtained from Merck as Mevacor® (see U.S. Pat. No. 4,231,938, the entire disclosure is herein incorporated by reference). Suitable pharmaceutically acceptable salts of lovastatin include one or more compounds derived from bases such as sodium hydroxide, potassium hydroxide, lithium hydroxide, calcium hydroxide, 1-deoxy-2-(methylamino)-D-glucitol, magnesium hydroxide, zinc hydroxide, aluminum hydroxide, ferrous or ferric hydroxide, ammonium hydroxide or organic amines such as N-methylglucamine, choline, arginine or the like or combinations thereof. Suitable pharmaceutically acceptable salts of lovastatin include lithium, calcium, hemicalcium, sodium, potassium, magnesium, aluminum, ferrous or ferric salts thereof or a combination thereof.

In various embodiments, the therapeutically effective amount of lovastatin comprises from about 0.1 pg to about 2000 mg, for example, 0.1 ng to 1000 mg, 500 mg, 100 mg, 50 mg, 25 mg, 10 mg, 1 mg, 50 mg, 25 mg, 10 mg, 1 mg, 500 ng, 250 ng, 100 ng, 75 ng, 50 ng, 25 ng, 15 ng, 10 ng, 5 ng, or 1 ng of lovastatin per day. In various embodiments, the dosage may be, for example from about 3 ng/day to 0.3 mg/day.

### Morphine

In one embodiment the analgesic is in liquid form and/or in the depot and comprises morphine. Morphine is also referred to as (5α,6α)-7,8-didehydro-4,5-epoxy-17-methylmorphinan-3,6-diol and has the chemical formula C17H19NO3. Morphine and a pharmaceutically acceptable salt thereof is available from various manufacturers. In one exemplary embodiment, the morphine comprises morphine sulfate or hydrochloride.

The dosage of the morphine may be from 0.1 mg to 1000 mg per day. For example, the dosage of morphine may be for example, 0.1 mg to 2 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 75 mg, 80 mg, 85 mg, 90 mg, 95 mg, 100 mg, 120 mg, 130 mg, 140 mg, 150 mg, 160 mg, 170 mg, 180 mg, 190 mg, 200 mg of morphine per day.

### Tramadol

In one embodiment, the analgesic is in liquid form and/or in the depot and comprises tramadol. Tramadol is also referred to as (±)cis-2-[(dimethylamino)methyl]-1-(3-methoxyphenyl)cyclohexanol hydrochloride and has the chemical formula C16H25NO2. Tramadol or a pharmaceutically acceptable salt thereof is available from various manufacturers. In various embodiments, tramadol HCL is used.

The dosage of the tramadol may be from 0.01 mg to 500 mg per day. For example, the dosage of tramadol may be for example, 0.1 mg to 2 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 75 mg, 80 mg, 85 mg, 90 mg, 95 mg, 100 mg, 120 mg, 130 mg, 140 mg, 150 mg, 160 mg, 170 mg, 180 mg, 190 mg, 200 mg, or 500 mg of tramadol per day.

In one embodiment, the drug depot contains sufficient tramadol to release between 2.5 and 30 mg/kg/day. In another embodiment the drug depot contains sufficient tramadol to release between 3 and 27.5 mg/kg/day.

In various embodiments, the active ingredient of the drug depot may have a high drug loading, such that the therapeutic agent comprises about 5-99 wt % of a depot, or 30-95 wt % of a depot, or 50-95 wt % of a depot. In some embodiments, the balance may be depot material, including optional inactive materials.

In some instance, it may be desirable to avoid having to remove the drug depot after use. In those instances, the depot may comprise a biodegradable material. There are numerous materials available for this purpose and having the characteristic of being able to breakdown or disintegrate over a prolonged period of time when positioned at or near the target tissue. As a function of the chemistry of the biodegradable material, the mechanism of the degradation process can be hydrolytical or enzymatical in nature, or both. In various embodiments, the degradation can occur either at the surface (heterogeneous or surface erosion) or uniformly throughout the drug delivery system depot (homogeneous or bulk erosion).

In various embodiments, the depot may comprise a bioabsorbable, a bioabsorbable, and/or a biodegradable biopolymer that may provide immediate release, or sustained release of the active ingredient. Examples of suitable sustained release biopolymers include but are not limited to poly (alpha-hydroxy acids), poly (lactide-co-glycolide) (PLGA), polylactide (PLA), polyglycolide (PG), polyethylene glycol (PEG) conjugates of poly (alpha-hydroxy acids), polyorthoesters, polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D,L-lactide, or L-lactide,-caprolactone, dextrans, vinylpyrrolidone, polyvinyl alcohol (PVA), PVA-g-PLGA, PEGT-PBT copolymer(polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate) or combinations thereof.

The depot may optionally contain inactive materials such as buffering agents and pH adjusting agents such as potassium bicarbonate, potassium carbonate, potassium hydroxide, sodium acetate, sodium borate, sodium bicarbonate, sodium carbonate, sodium hydroxide or sodium phosphate; degradation/release modifiers; drug release adjusting agents; emulsifiers; preservatives such as benzalkonium chloride, chlorobutanol, phenylmercuric acetate and phenylmercuric nitrate, sodium bisulfite, sodium bisulfate, sodium thiosulfate, thimerosal, methylparaben, polyvinyl alcohol and phenylethyl alcohol; solubility adjusting agents; stabilizers; and/or cohesion modifiers. Typically, any such inactive materials will be present within the range of 0-75 wt %, and more typically within the range of 0-30 wt %. If the depot is to be placed in the spinal area, in various embodiments, the depot may comprise sterile preservative free material.

The depot can be different sizes, shapes and configurations. There are several factors that can be taken into consideration in determining the size, shape and configuration of the drug depot. For example, both the size and shape may allow for ease in positioning the drug depot at the target tissue site that is selected as the implantation or injection site. In addition, the shape and size of the system should be selected so as to minimize or prevent the drug depot from moving after implantation or injection. In various embodiments, the drug depot can be shaped like a sphere, a cylinder such as a rod or fiber, a flat surface such as a disc, film or sheet (e.g., ribbon-like) and the like. Flexibility may be a consideration so as to facilitate placement of the drug depot. In various embodiments, the drug depot can be different sizes, for example, the drug depot may be a length of from about 0.5 mm to 5 mm and have a diameter of from about 0.01 to about 2 mm. In various embodiments, the drug depot may have a layer thickness of from about 0.005 to 1.0 mm, such as, for example, from 0.05 to 0.75 mm.

In various embodiments, when the drug depot comprises a ribbon-like fiber, it may be placed at the incision site before the site is closed. The ribbon-like fibers may for example be made of thermosplastic materials. Additionally, specific materials that may be advantageous for use as ribbon-like fibers include but are not limited to the compounds identified above as sustained release biopolymers. The ribbon-like fiber may be formed by mixing the clonidine with the polymer.

Radiographic markers can be included on the drug depot to permit the user to position the depot accurately into the target site of the patient. These radiographic markers will also permit the user to track movement and degradation of the depot at the site over time. In this embodiment, the user may accurately position the depot in the site using any of the numerous diagnostic imaging procedures. Such diagnostic imaging procedures include, for example, X-ray imaging or fluoroscopy. Examples of such radiographic markers include, but are not limited to, barium, calcium phosphate, and/or metal beads or particles. In various embodiments, the radiographic marker could be a spherical shape or a ring around the depot.

In various embodiments, the drug depot is in the form of a gel that has a pre-dosed viscosity in the range of about 1 to about 500 centipoise (cps), 1 to about 200 cps, or 1 to about 100 cps. After the gel is administered to the target site, the viscosity of the gel will increase and the gel will have a modulus of elasticity (Young's modulus) in the range of about 1×10⁴ to about 6×10⁵ dynes/cm², or 2×10⁴ to about 5×10⁵ dynes/cm², or 5×10⁴ to about 5×10⁵ dynes/cm².

In one embodiment, a depot comprises an adherent gel comprising clonidine that is evenly distributed throughout the gel. The gel may be of any suitable type, as previously indicated, and should be sufficiently viscous so as to prevent the gel from migrating from the targeted delivery site once deployed; the gel should, in effect, stick or adhere to the targeted tissue site. The gel may, for example, solidify upon contact with the targeted tissue or after deployment from a targeted delivery system. The targeted delivery system may be, for example, a syringe, a catheter, needle or cannula or any other suitable device. The targeted delivery system may inject the gel into or on the targeted tissue site. The therapeutic agent may be mixed into the gel prior to the gel being deployed at the targeted tissue site. In various embodiments, the gel may be part of a two-component delivery system and when the two components are mixed, a chemical process is activated to form the gel and cause it to stick or to adhere to the target tissue.

In various embodiments, a gel is provided that hardens or stiffens after delivery. Typically, hardening gel formulations may have a pre-dosed modulus of elasticity in the range of about 1×10⁴ to about 3×10⁵ dynes/cm², or 2×10⁴ to about 2×10⁵ dynes/cm², or 5×10⁴ to about 1×10⁵ dynes/cm². The post-dosed hardening gels (after delivery) may have a rubbery consistency and have a modulus of elasticity in the range of about 1×10⁴ to about 2×10⁶ dynes/cm², or 1×10⁵ to about 7×10⁵ dynes/cm², or 2×10⁵ to about 5×10⁵ dynes/cm².

In various embodiments, for those gel formulations that contain a polymer, the polymer concentration may affect the rate at which the gel hardens (e.g., a gel with a higher concentration of polymer may coagulate more quickly than gels having a lower concentration of polymer). In various embodiments, when the gel hardens, the resulting matrix is solid but is also able to conform to the irregular surface of the tissue (e.g., recesses and/or projections in bone).

The percentage of polymer present in the gel may also affect the viscosity of the polymeric composition. For example, a composition having a higher percentage by weight of polymer is typically thicker and more viscous than a composition having a lower percentage by weight of polymer. A more viscous composition tends to flow more slowly. Therefore, a composition having a lower viscosity may be preferred in some instances.

In various embodiments, the molecular weight of the gel can be varied by many methods known in the art. The choice of method to vary molecular weight is typically determined by the composition of the gel (e.g., polymer, versus non-polymer). For example in various embodiments, when the gel comprises one or more polymers, the degree of polymerization can be controlled by varying the amount of polymer initiators (e.g. benzoyl peroxide), organic solvents or activator (e.g. DMPT), crosslinking agents, polymerization agent, and/or reaction time.

Suitable gel polymers may be soluble in an organic solvent. The solubility of a polymer in a solvent varies depending on the crystallinity, hydrophobicity, hydrogen-bonding and molecular weight of the polymer. Lower molecular weight polymers will normally dissolve more readily in an organic solvent than high-molecular weight polymers. A polymeric gel, which includes a high molecular weight polymer, tends to coagulate or solidify more quickly than a polymeric composition, which includes a low-molecular weight polymer. Polymeric gel formulations, which include high molecular weight polymers, also tend to have a higher solution viscosity than a polymeric gel, which include a low-molecular weight polymer.

When the gel is designed to be a flowable gel, it can vary from low viscosity, similar to that of water, to a high viscosity, similar to that of a paste, depending on the molecular weight and concentration of the polymer used in the gel. The viscosity of the gel can be varied such that the polymeric composition can be applied to a patient's tissues by any convenient technique, for example, by brushing, dripping, injecting, or painting. Different viscosities of the gel will depend on the technique used to apply the composition.

In various embodiments, the gel has an inherent viscosity (abbreviated as “I.V.” and units are in deciliters/gram), which is a measure of the gel's molecular weight and degradation time (e.g., a gel with a high inherent viscosity has a higher molecular weight and longer degradation time). Typically, a gel with a high molecular weight provides a stronger matrix and the matrix takes more time to degrade. In contrast, a gel with a low molecular weight degrades more quickly and provides a softer matrix. In various embodiments, the gel has a molecular weight, as shown by the inherent viscosity, from about 0.10 dL/g to about 1.2 dL/g or from about 0.10 dL/g to about 0.40 dL/g.

In various embodiments, the gel can have a viscosity of about 300 to about 5,000 centipoise (cp). In other embodiments, the gel can have a viscosity of from about 5 to about 300 cps, from about 10 cps to about 50 cps, from about 15 cps to about 75 cps at room temperature. The gel may optionally have a viscosity enhancing agent such as, for example, hydroxypropyl cellulose, hydroxypropyl methylcellulose, hydroxyethyl methylcellulose, carboxymethylcellulose and salts thereof, Carbopol, poly(hydroxyethylmethacrylate), poly(methoxyethylmethacrylate), poly(methoxy-ethoxyethyl methacrylate), polymethylmethacrylate (PMMA), methylmethacrylate (MMA), gelatin, polyvinyl alcohols, propylene glycol, PEG 200, PEG 300, PEG 400, PEG 500, PEG 600, PEG 700, PEG 800, PEG 900, PEG 1000, PEG 1450, PEG 3350, PEG 4500, PEG 8000 or combinations thereof.

A gel with a higher viscosity may be desirable for certain applications, for example, a gel having a putty-like consistency may be more preferable for bone regeneration applications. In various embodiments, when a polymer is employed in the gel, the polymeric composition includes about 10 wt % to about 90 wt % or about 30 wt % to about 60 wt % of the polymer.

In various embodiments, the gel is a hydrogel made of high molecular weight biocompatible elastomeric polymers of synthetic or natural origin. A desirable property for the hydrogel to have is the ability to respond rapidly to mechanical stresses, particularly shears and loads, in the human body.

Hydrogels obtained from natural sources are particularly appealing because they are more likely to be biodegradable and biocompatible for in vivo applications. Suitable hydrogels include natural hydrogels, such as for example, gelatin, collagen, silk, elastin, fibrin and polysaccharide-derived polymers like agarose, and chitosan, glucomannan gel, hyaluronic acid, polysaccharides, such as cross-linked carboxyl-containing polysaccharides, or a combination thereof. Synthetic hydrogels include, but are not limited to those formed from polyvinyl alcohol, acrylamides such as polyacrylic acid and poly(acrylonitrile-acrylic acid), polyurethanes, polyethylene glycol (e.g., PEG 3350, PEG 4500, PEG 8000), silicone, polyolefins such as polyisobutylene and polyisoprene, copolymers of silicone and polyurethane, neoprene, nitrile, vulcanized rubber, poly(N-vinyl-2-pyrrolidone), acrylates such as poly(2-hydroxy ethyl methacrylate) and copolymers of acrylates with N-vinyl pyrolidone, N-vinyl lactams, polyacrylonitrile or combinations thereof. The hydrogel materials may further be cross-linked to provide further strength as needed. Examples of different types of polyurethanes include thermoplastic or thermoset polyurethanes, aliphatic or aromatic polyurethanes, polyetherurethane, polycarbonate-urethane or silicone polyether-urethane, or a combination thereof.

In various embodiments, rather than directly admixing the therapeutic agent into the gel, microspheres may be dispersed within the gel, the microspheres loaded with the active ingredient, e.g., clonidine. In one embodiment, the microspheres provide for a sustained release of the clonidine. In yet another embodiment, the gel, which is biodegradable, prevents the microspheres from releasing the clonidine; the microspheres thus do not release the clonidine until they have been released from the gel. For example, a gel may be deployed around a target tissue site (e.g., a nerve root). Dispersed within the gel are a plurality of microspheres that encapsulate the desired therapeutic agent. Certain of these microspheres degrade once released from the gel, thus releasing the active ingredient.

Microspheres, much like a fluid, may disperse relatively quickly, depending upon the surrounding tissue type, and hence disperse the clonidine. In some situations, this may be desirable; in others, it may be more desirable to keep the active ingredient tightly constrained to a well-defined target site. The present invention also contemplates the use of adherent gels to so constrain dispersal of the therapeutic agent. These gels may be deployed, for example, in a disc space, in a spinal canal, or in surrounding tissue.

Timing of Introduction

In certain embodiments, first the liquid formulation is introduced into body of the patient at or near the target site and next the drug depot is administered. The liquid formulation may be designed to provide immediate release upon administration. The drug depot may be part of a formulation that is designed to provide both immediate release and sustained release capabilities or it may be part of a formulation that is designed to provide only a sustained release.

Moreover, depending upon the formulation of the drug depot, the sustained release profile may begin after a certain amount of time, e.g., after the time at with the active ingredients from the liquid formulation no longer provide the desired result or shortly before they will no longer provide the desired result. For example, if the injectable formulation provided effective pain release for 24 hours, the drug depot formulation may be designed to begin its release at the same time the injectable formulation is introduced, after about two hours, after about four hours, after about six hours, after about eight hours, after about ten hours, after about twelve hours, after about fourteen hours, after about sixteen hours, after about eighteen hours, after about twenty hours, after about twenty-two hours or at about twenty-four hours.

The timing of the release of the active ingredient may also be designed to be dependant on the percentage of duration of the effect of the active ingredient. For example, the drug depot may be formulated to begin its release immediately after implantation, after about 10% of the period in which the active ingredient in the liquid formulation is effective, after about 20% of the period in which the active ingredient in the liquid formulation is effective, after about 30% of the period in which the active ingredient in the liquid formulation is effective, after about 40% of the period in which the active ingredient in the liquid formulation is effective, after about 50% of the period in which the active ingredient in the liquid formulation is effective, after about 60% of the period in which the active ingredient in the liquid formulation is effective, after about 70% of the period in which the active ingredient in the liquid formulation is effective, after about 80% of the period in which the active ingredient in the liquid formulation is effective, after about 90% of the period in which the active ingredient in the liquid formulation is effective, after about 95% of the period in which the active ingredient in the liquid formulation is effective, or after about 99% of the period in which the active ingredient in the liquid formulation is effective.

Because the release of the active ingredient of the drug depot may be controlled through for example the inclusion of certain polymers that are know to facilitate extended release of active ingredients, in some embodiments, one could implant the drug depot prior to administering the liquid formulation while maintaining the ability to commence the release of the active ingredient in the drug depot to begin at a desired time, including after the injection of the liquid formulation.

Additionally, in some embodiments, the drug depot is introduced into the patient at least 10 seconds after the introduction of the liquid formulation, at least 1 minute after the introduction of the liquid formulation, at least 1 hour after the introduction of the liquid formulation, at least 2 hours after the introduction of the liquid formulation, at least 3 hours after the introduction of the liquid formulation, at least 4 hours after the introduction of the liquid formulation. In some embodiments the drug depot is introduced into the organism less than 10 seconds after the introduction of the liquid formulation, less than 20 seconds after the introduction of the liquid formulation, less than 1 minute after the introduction of the liquid formulation, less than 5 minutes after the introduction of the liquid formulation, less than 10 minutes after the introduction of the liquid formulation, less than 15 minutes after the introduction of the liquid formulation, less than 20 minutes after the introduction of the liquid formulation, less than 25 minutes after the introduction of the liquid formulation, less than 30 minutes after the introduction of the liquid formulation, less than 35 minutes after the introduction of the liquid formulation, less than 40 minutes after the introduction of the liquid formulation, less than 45 minutes after the introduction of the liquid formulation, less than 55 minutes after the introduction of the liquid formulation, less than 60 minutes after the introduction of the liquid formulation, less than 1 hour after the introduction of the liquid formulation, less than 2 hours after the introduction of the liquid formulation, less than 3 hours after the introduction of the liquid formulation, or less than 4 hours after the introduction of the liquid formulation.

Cannula or Needle

It will be appreciated by those with skill in the art that the depot can be administered to the target site using a cannula or needle that can be a part of a drug delivery device e.g., a syringe, a gun drug delivery device, or any medical device suitable for the application of a drug to a targeted organ or anatomic region. The cannula or needle of the drug depot device is designed to cause minimal physical and psychological trauma to the patient.

Cannulas or needles include tubes that may be made from materials, such as for example, polyurethane, polyurea, polyether(amide), PEBA, thermoplastic elastomeric olefin, copolyester, and styrenic thermoplastic elastomer, steel, aluminum, stainless steel, titanium, metal alloys with high non-ferrous metal content and a low relative proportion of iron, carbon fiber, glass fiber, plastics, ceramics or combinations thereof. The cannula or needle may optionally include one or more tapered regions. In various embodiments, the cannula or needle may be beveled. The cannula or needle may also have a tip style vital for accurate treatment of the patient depending on the site for implantation. Examples of tip styles include, for example, Trephine, Cournand, Veress, Huber, Seldinger, Chiba, Francine, Bias, Crawford, deflected tips, Hustead, Lancet, or Tuohey. In various embodiments, the cannula or needle may also be non-coring and have a sheath covering it to avoid unwanted needle sticks.

The dimensions of the hollow cannula or needle, among other things, will depend on the site for implantation. For example, the width of the epidural space is only about 3-5 mm for the thoracic region and about 5-7 mm for the lumbar region. Thus, the needle or cannula, in various embodiments, can be designed for these specific areas. In various embodiments, the cannula or needle may be inserted using a transforaminal approach in the spinal foramen space, for example, along an inflammed nerve root and the drug depot implanted at this site for treating the condition. Typically, the transforaminal approach involves approaching the intervertebral space through the intervertebral foramina.

Some examples of lengths of the cannula or needle may include, but are not limited to, from about 50 to 150 mm in length, for example, about 65 mm for epidural pediatric use, about 85 mm for a standard adult and about 110 mm for an obese adult patient. The thickness of the cannula or needle will also depend on the site of implantation. In various embodiments, the thickness includes, but is not limited to, from about 0.05 to about 1.655. The gauge of the cannula or needle may be the widest or smallest diameter or a diameter in between for insertion into a human or animal body. The widest diameter is typically about 14 gauge, while the smallest diameter is about 22 gauge. In various embodiments the gauge of the needle or cannula is about 18 to about 22 gauge.

In various embodiments, like the drug depot and/or gel, the cannula or needle includes dose radiographic markers that indicate location at or near the site beneath the skin, so that the user may accurately position the depot at or near the site using any of the numerous diagnostic imaging procedures. Such diagnostic imaging procedures include, for example, X-ray imaging or fluoroscopy. Examples of such radiographic markers include, but are not limited to, barium, calcium phosphate, and/or metal beads or particles.

In various embodiments, the needle or cannula may include a transparent or translucent portion that can be visualizable by ultrasound, fluoroscopy, x-ray, or other imaging techniques. In such embodiments, the transparent or translucent portion may include a radiopaque material or ultrasound responsive topography that increases the contrast of the needle or cannula relative to the absence of the material or topography.

The drug depot, and/or medical device to administer the drug may be sterilizable. In various embodiments, one or more components of the drug depot, and/or medical device to administer the drug are sterilized by radiation in a terminal sterilization step in the final packaging. Terminal sterilization of a product provides greater assurance of sterility than from processes such as an aseptic process, which require individual product components to be sterilized separately and the final package assembled in a sterile environment.

Typically, in various embodiments, gamma radiation is used in the terminal sterilization step, which involves utilizing ionizing energy from gamma rays that penetrates deeply in the device. Gamma rays are highly effective in killing microorganisms, they leave no residues nor have sufficient energy to impart radioactivity to the device. Gamma rays can be employed when the device is in the package and gamma sterilization does not require high pressures or vacuum conditions, thus, package seals and other components are not stressed. In addition, gamma radiation eliminates the need for permeable packaging materials.

In various embodiments, electron beam (e-beam) radiation may be used to sterilize one or more components of the device. E-beam radiation comprises a form of ionizing energy, which is generally characterized by low penetration and high-dose rates. E-beam irradiation is similar to gamma processing in that it alters various chemical and molecular bonds on contact, including the reproductive cells of microorganisms. Beams produced for e-beam sterilization are concentrated, highly-charged streams of electrons generated by the acceleration and conversion of electricity. E-beam sterilization may be used, for example, when the drug depot is included in a gel.

Other methods may also be used to sterilize the depot and/or one or more components of the device, including, but not limited to, gas sterilization, such as, for example, with ethylene oxide or steam sterilization.

In various embodiments, a kit is provided that may include additional parts along with the drug depot and/or medical device combined together to be used to implant the drug depot (e.g., ribbon-like fibers), as well as the liquid formulation and/or a medical device such as a syringe for injecting it. The kit may include the drug depot device in a first compartment. The second compartment may include a canister holding the drug depot and any other instruments needed for the localized drug delivery. A third compartment may include gloves, drapes, wound dressings and other procedural supplies for maintaining sterility of the implanting process, as well as an instruction booklet. A fourth compartment may include additional cannulas and/or needles. A fifth compartment may include the liquid formulation. Each tool may be separately packaged in a plastic pouch that is radiation sterilized. A cover of the kit may include illustrations of the implanting procedure and a clear plastic cover may be placed over the compartments to maintain sterility and illustrations of how and where to administer the liquid formulation.

Drug Delivery

In various embodiments, a method for delivering therapeutic agents into a site of pain of a patient is provided, the method comprising inserting a syringe at or near a target site, dispensing a liquid formulation from the syringe, inserting a cannula at or near a target tissue site and implanting the drug depot at the target site beneath the skin of the patient and brushing, dripping, injecting, or painting the gel in the target site to hold or have the drug depot adhere to the target site. In this way unwanted migration of the drug depot away from the target site is reduced or eliminated.

In various embodiments, to administer the gel having the drug depot dispersed therein to the desired site, first the cannula or needle can be inserted through the skin and soft tissue down to the target tissue site and the gel administered (e.g., brushed, dripped, injected, or painted, etc.) at or near the target site. In those embodiments where the drug depot is separate from the gel, first the cannula or needle can be inserted through the skin and soft tissue down to the site of injection and one or more base layer(s) of gel can be administered to the target site. Following administration of the one or more base layer(s), the drug depot can be implanted on or in the base layer(s) so that the gel can hold the depot in place or reduce migration. If required a subsequent layer or layers of gel can be applied on the drug depot to surround the depot and further hold it in place. Alternatively, the drug depot may be implanted first and then the gel placed (e.g., brushed, dripped, injected, or painted, etc.) around the drug depot to hold it in place. By using the gel, accurate and precise implantation of a drug depot can be accomplished with minimal physical and psychological trauma to the patient. The gel also avoids the need to suture the drug depot to the target site reducing physical and psychological trauma to the patient.

In various embodiments, when the target site comprises a spinal region, a portion of fluid (e.g., spinal fluid, etc.) can be withdrawn from the target site through the cannula or needle first and then the depot administered (e.g., placed, dripped, injected, or implanted, etc.). The target site will re-hydrate (e.g., replenishment of fluid) and this aqueous environment will cause the drug to be released from the depot.

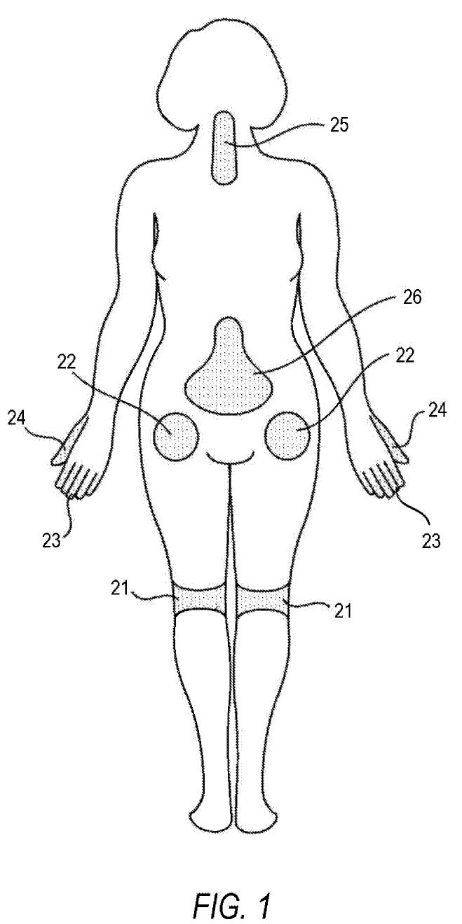

FIG. 1 illustrates a number of common locations within a patient that may be sites at which pain may occur, e.g., incidental to surgery. It will be recognized that the locations illustrated in FIG. 1 are merely exemplary of the many different locations within a patient that may be at which surgery took place. For example, surgery may be required at a patient's knees 21, hips 22, fingers 23, thumbs 24, neck 25, and spine 26. Thus, during or following these surgeries, the patient may be subject to muscle spasticity.

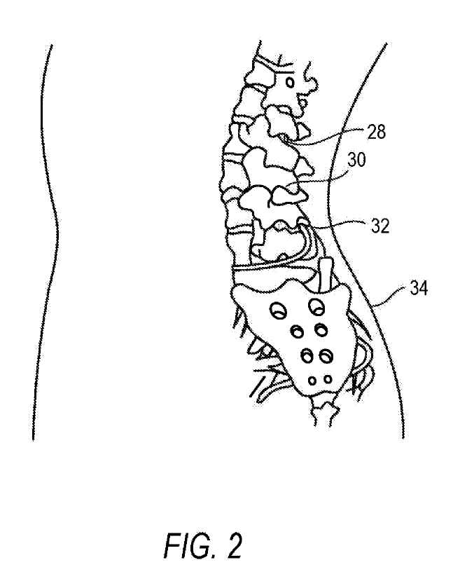

One exemplary embodiment where the liquid formulation and drug depot are suitable for use in pain management (e.g., neuropathic pain management) and/or to treat conditions (e.g., sciatica) is illustrated in FIG. 2. Schematically shown in FIG. 2 is a dorsal view of the spine and sites where the liquid formulation and drug depot may be inserted using a cannula or needle beneath the skin 34 to a spinal site 32 (e.g., spinal disc space, spinal canal, soft tissue surrounding the spine, nerve root, etc.) and one or more drug depots 28 and 32 are delivered to various sites along the spine. In this way, when the liquid formulation and several drug depots are to be implanted, they are implanted in a manner that optimizes location, accurate spacing, and drug distribution.

Although the spinal site is shown, as described above, the drug depot can be delivered to any site beneath the skin, including, but not limited to, at least one muscle, ligament, tendon, cartilage, spinal disc, spinal foraminal space, near the spinal nerve root, or spinal canal. Similarly, the liquid formulation can be administered at or near these locations. Further, in some embodiments, the liquid formulation is administered at a location closer to the target site than where the drug depot is placed.

In some embodiments, it is preferable to co-administer clonidine with an antagonist to counteract undesirable effects, for example the blood pressure decrease that can be caused by clonidine. Exemplary antagonists include but are not limited to phentolamine, yohimbine, tolazoline and piperoxane, which may be administered systemically in order to avoid blocking the local analgesic effects. Additionally, compounds such as 5-fluorodeoxyuridine (FUDR) and 3,4dehydroprolene may also be included. These compounds may prevent or reduce glial and fibroblastic scar formation associated with some types of surgeries.

In some embodiments, the therapeutically effective dosage amount and the release rate profile of the drug depot are sufficient to treat pain for a period of about 3-12 days; in other embodiments the release rate profile is sufficient to relax the muscle fibers for a period of about 5-10 days; in other embodiments the release rate profile is sufficient to relax the muscle fibers for a period of about 7-9 days. For example the release profile might be for a period of at least about 3 days, at least about 4 days, at least about 5 days, at least about 6 days, at least about 7 days, at least about 8 days, at least about 9 days, at least about 10 days, or at least about 11 days.

In some embodiments, the active ingredient of the drug depot is encapsulated in a plurality of depots comprising (micro)particles, (micro)spheres, (micro)capsules, and/or (micro)fibers.

In some embodiments, there is a liquid formulation comprising a steroid or clonidine or a combination thereof and an implantable drug depot, wherein the drug depot comprises: (i) one or more immediate release layers that release a bolus dose of clonidine or a pharmaceutically acceptable salt thereof at a site beneath the skin; and (ii) one or more sustain release layers that release an effective amount of clonidine or a pharmaceutically acceptable salt thereof over a period of about 3 to about 12 days or about 5 to about 10 days. The one or more immediate release layers may for example comprise poly(lactide-co-glycolide) (PLGA) and the one or more sustain release layers may for example comprise polylactide (PLA).

In some embodiments there is also method for making an implantable drug depot. The method may comprise combining a biocompatible polymer and a therapeutically effective amount of clonidine or a pharmaceutically acceptable salt thereof and forming the implantable drug depot from the combination.

In some embodiments, the clonidine is first compounded with a polymer to make a first component of the drug depot. In this first compounded product, the clonidine may for example, comprise 0.5% to 20% of the formulation by weight, 1% to 15% of the formulation by weight or 2.5% to 10% of the formulation by weight. The remainder of the formulation may be exclusively polymer or comprises excipients, surfactants or other inactive ingredients.

In some embodiments, the dosage of the clonidine in the drug depot is from approximately 0.0005 to approximately 100 μg/kg/day. Additional dosages of clonidine can include from approximately 0.0005 to approximately 95 μg/kg/day; approximately 0.0005 to approximately 90 μg/kg/day; approximately 0.0005 to approximately 85 μg/kg/day; approximately 0.0005 to approximately 80 μg/kg/day; approximately 0.0005 to approximately 75 μg/kg/day; approximately 0.001 to approximately 70 μg/kg/day; approximately 0.001 to approximately 65 μg/kg/day; approximately 0.001 to approximately 60 μg/kg/day; approximately 0.001 to approximately 55 μg/kg/day; approximately 0.001 to approximately 50 μg/kg/day; approximately 0.001 to approximately 45 μg/kg/day; approximately 0.001 to approximately 40 μg/kg/day; approximately 0.001 to approximately 35 μg/kg/day; approximately 0.0025 to approximately 30 μg/kg/day; approximately 0.0025 to approximately 25 μg/kg/day; approximately 0.0025 to approximately 20 μg/kg/day; and approximately 0.0025 to approximately 15 μg/kg/day. In another embodiment, the dosage of clonidine is from approximately 0.005 to approximately 15 μg/kg/day. In another embodiment, the dosage of clonidine is from approximately 0.005 to approximately 10 μg/kg/day. In another embodiment, the dosage of clonidine is from approximately 0.005 to approximately 5 μg/kg/day. In another embodiment, the dosage of clonidine is from approximately 0.005 to approximately 20 μg/kg/day. In some embodiments, the dose is 200 to 600 mg per day and is applied to a human.

In some embodiments it is desirable to use a sufficient amount of clonidine in the drug depot to be detected in the blood at least 1 microgram per kilogram of bodyweight, and preferably in an amount of 2-8 micrograms per kilogram, and even more preferably 3-6 micrograms per kilogram of bodyweight. Further, in some embodiments, it is desirable to administer a sufficient amount of clonidine in a single sustained release formulation to sustain this level for up to 12 days, e.g., 5-12 days or 7-10 days. In some embodiments, it is desirable for there to be less than or equal to 0.1 ng/mL of clonidine.

In some embodiments, the drug depot is administered parenterally, e.g., by injection. The term “parenteral” as used herein refers to modes of administration, which bypass the gastrointestinal tract, and include for example, localized intravenous, intramuscular, continuous or intermittent infusion, intraperitoneal, intrasternal, subcutaneous, intra-operatively, intrathecally, intradiscally, peridiscally, epidurally, perispinally, intraarticular injection or combinations thereof.

In some embodiments, the injection is intrathecal or epidural, which refers to an injection into the spinal canal (intrathecal space surrounding the spinal cord). An injection may also be into a muscle or other tissue. In other embodiments, the clonidine is adminstered by placement into an open patient cavity during surgery itself.

In some embodiments, a patient is first administered a contrast agent in combination with a steriod and local anlagesic through an epidural injection, followed by the administration of a local analgesic.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. A method for treating a mammal suffering from pain, the method comprising injecting a therapeutically effective amount of a liquid at or near a target site beneath the skin, wherein said liquid comprises a first active ingredient that is effective at treating pain, and after injecting said liquid, administering a drug depot locally at or near the target site, wherein the drug depot comprises a therapeutically effective amount of a second active ingredient that is effective at treating pain for at least 3 days.

2. A method according to claim 1, wherein the first active ingredient that is an immediate release liquid and the second active ingredient is a sustained release drug depot containing the same active ingredient.

3. A method according to claim 1, wherein the first active ingredient and the second active ingredient are the different.

4. A method according to claim 1, wherein the first active ingredient is administered locally at or near the target site and the first active ingredient is a steroid.

5. A method according to claim 1, wherein the first active ingredient is an immediate release liquid and comprises clonidine.

6. A method according to claim 1, wherein the second active ingredient is clonidine.

7. A method according to claim 4, wherein the second active ingredient is clonidine.

8. A method according to claim 5, wherein the second active ingredient is clonidine.

9. A method according to claim 6, wherein the drug depot is a pellet.

10. A method according to claim 1, wherein said drug depot releases clonidine for a period of at least 5 days.

11. A method according to claim 10, wherein said drug depot releases clonidine for a period of at least 7 days.

12. A method according to claim 11, wherein said therapeutically effective amount of said release is sufficient to release therapeutic amounts of clonidine for a period of at least 9 days.

13. A method according to claim 1, wherein the pain is at least one of acute radicular pain, neuropathic pain or inflammatory pain.

14. A method according to claim 1, wherein the pain is caused by tendonitis.

15. A method according to claim 4, wherein the steroid is a corticosteroid.

16. A method according to claim 4, wherein the steroid is a glucocorticoid.

17. A method according to claim 4, wherein from about 10 micrograms to about 160 micrograms of steroid are injected at or near the target site.

18. A method according to claim 5, wherein the clonidine is in the form of a solution containing about 10 to about 40 micrograms of clonidine hydrochloride.

19. A method according to claim 6, wherein the amount of clonidine is from about 200 to about 400 micrograms.

20. A method for treating a mammal suffering from pain, said method comprising injecting a therapeutically effective amount of a liquid at or near a target site, wherein said liquid comprises clonidine and a steroid that is effective at treating pain, and after injecting said liquid, injecting a drug depot locally at or near the target site, wherein the drug depot comprises a therapeutically effective amount of clonidine in a formulation that provides for relief from pain for a period of at least three days.
